(19) United States
(12) Patent Application Publication
(10) Pub. No.: US 2002/0132780 A1
(43) Pub. Date: Sep. 19, 2002

(54) Low carbohydrate compositions, kits thereof, and methods of use

(76) Inventors: Matthew Thomas Heisey, Wyoming, OH (US); Kenneth Norman Kern, Cincinnati, OH (US); Kris Eugene Spence, Madeira, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 09/759,965

(22) Filed: Jan. 12, 2001

(51) Int. Cl. A61K 38/39; A61K 31/737; A61K 31/7008; A61K 31/70; A61K 31/10

(52) U.S. Cl. 514/23; 514/21; 514/54; 514/62; 514/562; 514/708; 514/46; 514/53

(57) ABSTRACT

The present invention relates to compositions, kits, and methods utilized for the treatment of joint dysfunction, bone dysfunction, and/or inflammation. The composition utilized herein are useful for those mammals experiencing painful or debilitating joint, bone, or inflammatory conditions, and are particularly suited for mammals which are diabetic or at risk for diabetes, as well as those desiring or requiring conveniently dosed chondroprotective compositions having low carbohydrate content, low caloric value and/or having a low glycemic index.

In particular, the present compositions comprise:

a) a chondroprotective agent selected from gelatin, cartilage, aminosugars, glycosaminoglycans, methylsulfonylmethane, precursors of methylsulfonylmethane, S-adenosylmethionine, and mixtures thereof;

b) a sweetening agent other than glucose, dextrose, sucrose, and fructose; and

c) at least about 10% water, by weight of the composition.

In an alternative embodiment of the present invention, the present compositions comprise:

a) a chondroprotective agent selected from gelatin, cartilage, aminosugars, glycosaminoglycans, methylsulfonylmethane, precursors of methylsulfonylmethane, S-adenosylmethionine, salts thereof, and mixtures thereof; and

b) a sweetening agent other than glucose, dextrose, sucrose, and fructose;

wherein the composition is substantially free of aspartame.

Other compositions of the present invention comprise a chondroprotective agent selected from gelatin, cartilage, aminosugars, glycosaminoglycans, methylsulfonylmethane, precursors of methylsulfonylmethane, S-adenosylmethionine, and mixtures thereof, and have a low carbohydrate content, as defined herein.

### FIELD OF THE INVENTION

The present invention is directed to compositions which are useful for promoting one or more health benefits including, for example, joint health, bone health, and/or anti-inflammation. The present invention is further directed to kits comprising the compositions and methods of using the compositions and kits.

### BACKGROUND OF THE INVENTION

Osteoarthritis is a widespread, degenerative disease of the joints, cartilage, and other articular components. Osteoarthritis affects all ethnic groups worldwide. In addition to humans, osteoarthritis affects nearly all mammals, for example, horses and cows, as well as domestic cats and dogs. Many treatments for osteoarthritis have been proposed, all resulting in varying degrees of success.

One osteoarthritis treatment which has been recently proposed is oral administration of chondroprotective agents such as glucosamine and/or chondroitin. See e.g., Henderson, U.S. Pat. No. 5,364,845, assigned to Nutramax Laboratories, issued Nov. 15, 1994. Indeed, various commercial products are available in the marketplace, including nutritional supplements containing such agents and powders which may be formulated into beverage compositions immediately prior to use.

Typically, administration of such agents is designed to enhance proteoglycan through an increased concentration of glycosaminoglycans. Enhanced proteoglycan provides the framework for collagen and other joint components, as well as imparting flexibility, resiliency, and resistance to compression. Thus, these agents may be administered according to various methods to enhance the articular components or, at a minimum, inhibit the process of degradation.

Chondroprotective agents may be delivered in the form of compositions having a high sugar content (e.g., fruit juices) in order to improve compliance through improved palatability or convenience of the agent. However, these highly sugared compositions are extremely caloric and can contribute to the elevation of blood glucose levels. These may be undesirable for the ordinary consumer, and may be particularly undesirable for those individuals who are overweight, obese, or even those who merely understand the importance of limiting caloric and sugar intake.

Additionally, the diabetic individual may be precluded from ingesting such highly sugared compositions due to their potential affect on systemic blood glucose levels. There is also evidence that certain chondroprotective agents may actually adversely affect stabilization of blood glucose levels. Ironically, however, the diabetic individual will often have the most critical need for these chondroprotective agents. This critical need is, in part, due to the excess weight gain typically experienced by the diabetic individual, which can lead to increased pressure on the joints, cartilage and other articular components, and ultimately osteoarthritis.

As set forth herein, the present inventors have overcome the foregoing limitations of the previous chondroprotective compositions. The present inventors provide herein compositions comprising one or more chondroprotective agents in a convenient, palatable form which not only increases compliance, but is well-suited for those in need of a decreased caloric and/or sugar intake. Thus, the present compositions are surprisingly suitable for the broad spectrum of mammals which experience osteoarthritis worldwide. These and other benefits of the present invention are described in further detail herein.

### SUMMARY OF THE INVENTION

The present invention relates to compositions, kits, and methods utilized for the treatment of joint dysfunction, bone dysfunction, and/or inflammation. The compositions utilized herein are useful for those mammals experiencing painful or debilitating joint, bone, or inflammatory conditions, and are particularly suited for mammals which are diabetic or at risk for diabetes, as well as those desiring or requiring conveniently dosed chondroprotective compositions having low carbohydrate content, low caloric value and/or having a low glycemic index.

In particular, the present compositions comprise:

a) a chondroprotective agent selected from the group consisting of gelatin, cartilage, aminosugars, glycosaminoglycans, methylsulfonylmethane, precursors of methylsulfonylmethane, S-adenosylmethionine, and mixtures thereof;

b) a sweetening agent other than glucose, dextrose, sucrose, and fructose; and

c) at least about 10% water, by weight of the composition.

In an alternative embodiment of the present invention, the present compositions comprise:

a) a chondroprotective agent selected from the group consisting of gelatin, cartilage, aminosugars, glycosaminoglycans, methylsulfonylmethane, precursors of methylsulfonylmethane, S-adenosylmethionine, salts thereof, and mixtures thereof; and

b) a sweetening agent other than glucose, dextrose, sucrose, and fructose;

wherein the composition is substantially free of aspartame.

In another alternate embodiment of the present invention, the compositions comprise:

a) a chondroprotective agent selected from the group consisting of gelatin, cartilage, aminosugars, glycosaminoglycans, methylsulfonylmethane, precursors of methylsulfonylmethane, S-adenosylmethionine, salts thereof, and mixtures thereof;

b) at least about 10% water, by weight of the composition; and

c) less than about 18 grams total carbohydrate per every 230 milliliters of the composition.

In yet another embodiment of the present invention, the present compositions comprise:

a) a chondroprotective agent selected from the group consisting of gelatin, cartilage, aminosugars, glycosaminoglycans, methylsulfonylmethane, precursors of methylsulfonylmethane, S-adenosylmethionine, salts thereof, and mixtures thereof; and

b) less than about 18 grams total carbohydrate per every 230 milliliters of the composition;

wherein the composition is substantially free of aspartame.

The present invention is further directed to food, beverage, pharmaceutical, over-the-counter, and dietary supplement products which comprise the present compositions. The products are suitable for mammalian use. The invention also relates to kits comprising the present compositions and information that use of the composition promotes one or more of the presently defined health benefits, including joint health, bone health, and anti-inflammation. The present invention additionally relates to methods of treating joint function, bone function, or inflammation comprising administering to a mammal a composition as defined herein.

### DETAILED DESCRIPTION OF THE INVENTION

The present invention is directed to compositions which are useful, for example, in food, beverage, pharmaceutical, over-the-counter, and dietary supplement products. The food and beverage products include those which are traditional, as well as those which may be classified as “medical foods” under regulatory guidelines. The compositions are suitable for mammalian use, particularly use in humans and domestic animals such as, for example, dogs, cats, horses, and cows. The present invention is further directed to kits comprising such compositions and methods of using such compositions.

The compositions of the present invention are useful for providing one or more joint health, bone health, and/or anti-inflammation benefits. Joint health benefits include, but are not limited to, preventing, inhibiting, ceasing and/or reversing the symptoms and/or manifestations of arthritis, particularly osteoarthritis. Thus, improved joint health will provide, for example, inhibition of articular component degradation, decreased pain in the joints and/or increased flexibility. Bone health benefits include, but are not limited to, preventing, inhibiting, ceasing, and/or reversing bone loss and/or building bone mass, and/or preventing, inhibiting, ceasing, and/or reversing osteoporosis. Thus, improved bone health may provide, for example, healthy bones, stronger bones, and/or increased bone mass. Anti-inflammation benefits include, for example, preventing, inhibiting, ceasing, and/or reversing inflammation, particularly in the joints. Thus, anti-inflammation will typically result in pain reduction.

Publications and patents are referred to throughout this disclosure. All references cited herein are hereby incorporated by reference.

All percentages and ratios are calculated by weight unless otherwise indicated. All percentages and ratios are calculated based on the total composition unless otherwise indicated.

All component or composition levels are in reference to the active level of that component or composition, and are exclusive of impurities, for example, residual solvents or by-products, which may be present in commercially available sources.

Referred to herein are trade names for components including various ingredients utilized in the present invention. The inventors herein do not intend to be limited by materials under a certain trade name. Equivalent materials (e.g., those obtained from a different source under a different name or catalog (reference) number) to those referenced by trade name may be substituted and utilized in the compositions, kits, and methods herein.

As used herein, the total amount of any given component includes any added component as well as any of the component inherently present in the composition by virtue of inclusion of additional ingredients in the composition.

In the description of the invention various embodiments and/or individual features are disclosed. As will be apparent to the ordinarily skilled practitioner, all combinations of such embodiments and features are possible and can result in preferred executions of the present invention.

The compositions, kits, and methods herein may comprise, consist essentially of, or consist of any of the elements as described herein.

### Compositions of the Present Invention

The present invention is useful for those mammals experiencing painful or debilitating joint, bone, or inflammatory conditions, and is particularly suited for mammals which are diabetic or at risk for diabetes, as well as those desiring or requiring conveniently dosed chondroprotective compositions having low carbohydrate content, low caloric value and/or having a low glycemic index.

The compositions described herein are useful in, for example, food, beverage, pharmaceutical, over-the-counter, and dietary supplement products. The products are suitable for mammalian use, particularly use in humans and domestic animals such as, for example, dogs, cats, horses, and cows. Preferably, the present compositions are directed for use in humans and domestic animals. More preferably, the present compositions are directed for use in humans, domestic dogs, and domestic cats. Most preferably, the present compositions are directed for use in humans.

In accordance certain embodiments of the present invention, the present compositions comprise:

a) a chondroprotective agent selected from the group consisting of gelatin, cartilage, aminosugars, glycosaminoglycans, methylsulfonylmethane, precursors of methylsulfonylmethane, S-adenosylmethionine, salts thereof, and mixtures thereof;

b) a sweetening agent other than glucose, dextrose, sucrose, and fructose; and

c) at least about 10% water, by weight of the composition.

In other embodiments of the present invention, the present compositions comprise:

a) a chondroprotective agent selected from the group consisting of gelatin, cartilage, aminosugars, glycosaminoglycans, methylsulfonylmethane, precursors of methylsulfonylmethane, S-adenosylmethionine, salts thereof, and mixtures thereof; and

b) a sweetening agent other than glucose, dextrose, sucrose, and fructose;

wherein the composition is substantially free of aspartame.

Preferably, the compositions herein are low in carbohydrate content. Preferably, the compositions have a carbohydrate content of less than about 19 grams total carbohydrate per every 230 milliliters of the composition. More preferably, the compositions have a carbohydrate content of less than about 18.5 grams total carbohydrate per every 230 milliliters of the composition. Even more preferably, the compositions have a carbohydrate content of less than about 18 grams total carbohydrate per every 230 milliliters of the composition. Most preferably, the compositions have a carbohydrate content of less than about 17.5 grams total carbohydrate per every 230 milliliters of the composition. As used herein, “carbohydrate content” is determined by “difference”, according to the standard art method, by determining the total amino acid content, moisture content, lipid content, and ash content of a given composition to be measured. The remainder of such composition is determined to be the total carbohydrate of the composition, with the term “carbohydrate content” being expressed as total carbohydrate per every 230 milliliters of the composition.

Therefore, in another embodiment of the present invention, the compositions comprise:

a) a chondroprotective agent selected from the group consisting of gelatin, cartilage, aminosugars, glycosaminoglycans, methylsulfonylmethane, precursors of methylsulfonylmethane, S-adenosylmethionine, salts thereof, and mixtures thereof;

b) at least about 10% water, by weight of the composition; and

c) less than about 19 grams total carbohydrate per every 230 milliliters of the composition.

In other embodiments of the present invention, the present compositions comprise:

a) a chondroprotective agent selected from the group consisting of gelatin, cartilage, aminosugars, glycosaminoglycans, methylsulfonylmethane, precursors of methylsulfonylmethane, S-adenosylmethionine, salts thereof, and mixtures thereof; and

b) less than about 19 grams total carbohydrate per every 230 milliliters of the composition;

wherein the composition is substantially free of aspartame.

The various components of the present compositions are described further herein. The compositions are particularly suited for treating joint function, bone function, and/or inflammation in a mammal. Most preferably, the compositions are useful for treating joint function and inflammation in a mammal, particularly joint function.

The Chondroprotective Agent

The chondroprotective agent utilized in the present compositions is selected from the group consisting of gelatin, cartilage, aminosugars, glycosaminoglycans, methylsulfonylmethane, precursors of methylsulfonylmethane, S-adenosylmethionine, and mixtures thereof. This component is a key component particularly useful for providing bone health, joint health, and anti-inflammation, most preferably joint health.

Without intending to be limited by theory, the chondroprotective agent is important for enhancing joint function as the component may aid in the stimulation of proteoglycan and collagen in vivo. Proteoglycan provides the connective tissue, for example, collagen, which is necessary for joint health. Indeed, proteoglycan is comprised of glycosaminoglycans (often termed “GAGs”) which are long chains of modified sugars. Aminosugars and methylsulfonylmethane are useful for building glycosaminoglycans and proteoglycan.

Preferably, the chondroprotective agent is selected from gelatin, cartilage, aminosugars, glycosaminoglycans, S-adenosylmethionine, and mixtures thereof. More preferably, the chondroprotective agent is selected from aminosugars, glycosaminoglycans, S-adenosylmethionine, salts thereof, and mixtures thereof. Even more preferably, the chondroprotective agent is selected from aminosugars, glycosaminoglycans, and mixtures thereof. Most preferably, the chondroprotective agent is a salt of an aminosugar, particularly wherein the aminosugar is glucosamine.

The various chondroprotective agents, and preferred embodiments thereof, are described in expanded detail as follows. Typically, the present compositions comprise from about 0.0001% to about 75%, preferably from about 0.001% to about 50%, more preferably from about 0.01% to about 20%, even more preferably from about 0.1% to about 10%, and most preferably from about 0.1% to about 2% of the chondroprotective agent, all by weight of the composition, depending on the final form of the composition. For example, ready-to-drink beverage compositions will more typically comprise from about 0.0001% to about 10%, more preferably from about 0.001% to about 2%, and most preferably from about 0.01% to about 1% of the chondroprotective agent, by weight of the composition. Dry beverage compositions (i.e., those which are suitable for dilution to provide a concentrate or ready-to-drink beverage composition) will more typically comprise from about 1% to about 75%, more preferably from about 2% to about 50%, even more preferably from about 5% to about 25%, and most preferably from about 15% to about 20% of the chondroprotective agent, by weight of the composition.

Alternatively, appropriate levels of chondroprotective agent may be expressed as a mass dose, as set forth individually for each of the preferred chondroprotective agents listed below. With respect to dosing preferences, all dosage levels are based on typical human subjects (e.g., about a 55 to 65 kg subject). Wherein the present composition is used in other mammals, it may be necessary to modify the dosage. Modification of dosages based on the needs of the subject is well within the skill of the ordinary artisan. It therefore understood that these dosage ranges are by way of example only, and that daily administration can be adjusted depending on various factors. The specific dosage of the chondroprotective agent to be administered, as well as the duration of treatment are interdependent. The dosage and treatment regimen will also depend upon such factors as the specific chondroprotective agent used, the treatment indication, the efficacy of the compound, the personal attributes of the subject (such as, for example, weight, age, sex, and medical condition of the subject), and compliance with the treatment regimen.

Gelatin

As is commonly known, gelatin is a protein obtained from the partial hydrolysis of collagen, which is the major structural and connective protein tissue in mammals. Gelatin typically contains from about 84% to about 90% protein, from about 1% to about 2% mineral salts, and from about 8% to about 15% water (these are non-limiting approximations). Gelatin typically contains specific amounts of 18 different amino acids, which are joined together to form polypeptide chains of approximately 1,000 amino acid residues per chain.

Typically, the collagen obtained for gelatin production is from animal bones and skins, e.g., from cows and pigs. Gelatin production will typically involve the subjection of collagenous material to alkaline pre-treatment, followed by hot-water extraction (providing gelatin having an iso-electric point of about 5). Acidic pre-treatment may also be utilized (providing gelatin having an iso-electric point of from about 7 to 9).

In accordance with the present invention, wherein gelatin is included within a present composition, a single dose of gelatin within the composition is preferably from about 1 mg to about 2000 mg, more preferably from about 100 mg to about 700 mg, even more preferably from about 150 mg to about 600 mg, and most preferably from about 200 mg to about 400 mg. Typically, the composition comprising gelatin is dosed from about once to about five times daily. However, in the food and beverage composition embodiments of the present invention, which are preferred, a typical dosage can be increased accordingly such that dosing need only occur about once daily. Thus, in these food and beverage compositions, compliance and consumer benefit is enhanced.

Cartilage

Cartilage, including hydrolyzed cartilage, may be chosen as the chondroprotective agent in the present compositions. As is commonly known in the art, cartilage is a tough, elastic tissue present in the joints (as well as other locations) of the bodies of various mammals. Cartilage is comprised of at least one of calcium, proteins, carbohydate mucopolysaccharides, and collagen.

Particularly preferred for use herein is bovine cartilage and shark cartilage. Bovine cartilage is primarily derived from the trachea of cows (also known as bovine tracheal cartilage, or BTC). It is similar in structure to shark cartilage. Shark cartilage is a widely utilized cartilage source, as the skeletons of sharks are primarily composed of cartilage rather than bone.

In accordance with the present invention, wherein cartilage is included within a present composition, a single dose of cartilage within the composition is preferably from about 1 mg to about 2000 mg, more preferably from about 100 mg to about 700 mg, even more preferably from about 150 mg to about 600 mg, and most preferably from about 200 mg to about 400 mg. Typically, the composition comprising cartilage is dosed from about once to about five times daily. However, in the food and beverage composition embodiments of the present invention, which are preferred, a typical dosage can be increased accordingly such that dosing need only occur about once daily.

Aminosugars

One or more aminosugars may be chosen as the chondroprotective agent herein. The aminosugars are monosaccharide components (i.e., hexoses) which are modified with an amine functionality. The amine functionality may be a free amine moiety or a protected amine moiety (e.g., N-acetyl amine). Preferably, the aminosugar is a precursor to glycosaminoglycan, which is important for construction of joint constituents (e.g., collagen). Additionally, certain aminosugars may inhibit the activity of enzymes which are implicated in breakdown the cartilage in osteoarthritics (e.g., mannosamine, which has been discovered to inhibit aggrecanase). The aminosugars are well-known in the art; many aminosugars are naturally occurring.

Particularly preferred aminosugars include glucosamine, salts of glucosamine, galactosamine, salts of galactosamine, mannosamine, salts of mannosamine, as well as the N-acetyl derivatives of the foregoing, including N-acetyl glucosamine and N-acetyl galactosamine. More preferably, the aminosugars include glucosamine and salts of glucosamine, most preferably salts of glucosamine. Particularly preferred salts of glucosamine include glucosamine sulfate and glucosamine hydrochloride. The salts of glucosamine are particularly preferred to aid bioavailability of the aminosugar.

As an example, glucosamine provides the building block needed in vivo to manufacture glycosaminoglycan, which is found in cartilage. Thus, glucosamine, and other aminosugars, function not only to relieve symptoms of joint pain but also stop, inhibit, and/or reverse the degenerative process.

Typical single dosing of the aminosugars is preferably from about 1 mg to about 5000 mg, more preferably from about 100 mg to about 3600 mg, even more preferably from about 150 mg to about 2200 mg, and most preferably from about 250 mg to about 1900 mg, based on the molecular weight of glucosamine hydrochloride. For example, a particularly preferred dosage of glucosamine hydrochloride is about 1800 mg, which translates to about 1480 mg of glucosamine. All other aminosugars may be similarly dosed, based on the molecular weight of glucosamine hydrochloride. Typically, the composition comprising the aminosugar is dosed from about once to about five times daily, preferably from about once to about three times daily. However, in the food and beverage composition embodiments of the present invention, which are preferred, a typical dosage can be increased accordingly such that dosing need only occur about once daily.

Glycosaminoglycans

One or more glycosaminoglycans may be utilized as the chondroprotective agent herein. The glycosaminoglycans are commonly known as GAGs, and are precursors to joint structure, for example, collagen. The glycosaminoglycans may also be important for the healing of bone.

Suitable glycosaminoglycans will be well-known to the ordinarily skilled artisan. Preferred glycosaminoglycans include chondroitin, hyaluronic acid, keratan, heparin, and dermatin, as well as salts of the foregoing. For example, chondroitin sulfate is a particularly preferred chondroitin salt. As with the aminosugars, salts of the glycosaminoglycans are particularly preferred for use herein.

As an example, chondroitin provides the structure and allows various molecules to transport through cartilage (which is important, since there is no blood supply to cartilage). Chondroitin is a major constituent of cartilage and contains repeating chains of mucopolysaccharides.

Typical single dosing of the glycosaminoglycans is preferably from about 1 mg to about 10 grams, more preferably from about 100 mg to about 5 grams, even more preferably from about 150 mg to about 1000 mg, and most preferably from about 250 mg to about 800 mg, based on the molecular weight of chondroitin. All other glycosaminoglycans may be similarly dosed, based on the molecular weight of chondroitin. Typically, the composition comprising the glycosaminoglycan is dosed from about once to about five times daily. However, in the food and beverage composition embodiments of the present invention, which are preferred, a typical dosage can be increased accordingly such that dosing need only occur about once daily.

Methylsulfonylmethane and Precursors of Methylsulfonylmethane

The chondroprotective agent herein may also be methylsufonylmethane, or a precursor thereof. As used herein, the term “precursor thereof” means a compound which, in mammalian systems, is converted to methylsulfonylmethane in vivo. Methylsulfonylmethane, and precursors thereof, are common ingredients found in vivo and in nature, e.g., in unprocessed foods. Without intending to be limited by theory, it is believed that the sulfur moiety present in methylsulfonylmethane, and its precursors, provides the disulfide bridging (also commonly known as “tie-bars” or “cross-links”) necessary to hold the connective tissue in joints together.

While unprocessed foods contain methylsulfonylmethane, and the precursors thereof, conventional food processing and preparation causes the loss of these compounds from the foods. Therefore, commonly ingested foods may become deficient in these compounds. In these respects, methylsulfonylmethane is similar to vitamins and minerals which are typically partially or totally lost during normal food processing and preparation. It is therefore an important embodiment of this invention to include, as the chondroprotective agent, methylsulfonylmethane or a precursor thereof in the present compositions.

Non-limiting examples of precursors of methylsulfonylmethane include methionine and methyl sulfide. See e.g., Herschler et al., U.S. Pat. No. 4,863,748, issued Sep. 5, 1989. Precursors of methylsulfonylmethane is associated with a variety of health benefits, including joint benefits (such as relief from osteoarthritis and rheumatoid arthritis), as well as anti-inflammation.

In accordance with the present invention, wherein methanesulfonylmethane is included within a present composition, a single dose of methanesulfonylmethane within the composition is preferably from about 0.01 mg to about 2000 mg, more preferably from about 0.01 mg to about 500 mg, even more preferably from about 1 mg to about 200 mg, and most preferably from about 1 mg to about 100 mg. The precursors of methanesulfonylmethane may be similarly dosed, based on the molecular weights of the precursors relative to methanesulfonylmethane. Typically, the composition comprising methanesulfonylmethane is dosed from about once to about five times daily. However, in the food and beverage composition embodiments of the present invention, which are preferred, a typical dosage can be increased accordingly such that dosing need only occur about once daily.

S-Adenosylmethionine

S-adenosylmethionine, which is commonly known as “SAM-e,” is a compound which is found in most, if not all, living cells. Without intending to be limited by theory, SAM-e is produced through reaction of the essential amino acid methionine and the energy molecule known as adenosine triphosphate (commonly known as ATP). SAM-e manufactures the components of cartilage and repairs, restores, and maintains joint function. SAM-e is made in vivo from the amino acid methionine, and is found in ordinary dietary sources such as meats, soybeans, eggs, seeds, and lentils.

In accordance with the present invention, wherein SAM-e is included within a present composition, a single dose of SAM-e within the composition is preferably from about 1 mg to about 2000 mg, more preferably from about 100 mg to about 700 mg, even more preferably from about 150 mg to about 600 mg, and most preferably from about 200 mg to about 400 mg. Typically, the composition comprising SAM-e is dosed from about once to about five times daily. However, in the food and beverage composition embodiments of the present invention, which are preferred, a typical dosage can be increased accordingly such that dosing need only occur about once daily.

The Sweetening Agent

The sweetening agent, as defined herein, is a sweetener other than glucose, dextrose, sucrose, and fructose. However, the present inventors do not intend to exclude the use of one or more of glucose, dextrose, sucrose, or fructose, provided that the glucose, dextrose, sucrose, and/or fructose is utilized in addition to the defined sweetening agent (see herein below regarding “caloric sweeteners”).

Sweetening agents are commonly known in the art. As stated herein, sweetening agents which provide little to no caloric value in the composition, as well as a low glycemic index, are particularly preferred for use in the present compositions. Non-limiting examples of such sweetening agents include sorbitol, mannitol, xylitol, erythritol, malitol, maltose, lactose, fructooligosaccharides, lo han guo, stevioside, acesulfame, aspartame, sucralose, saccharin, xylose, arabinose, levulose, isomalt, ribose, and mixtures thereof. Preferred among these examples include xylitol, erythritol, fructooligosaccharides, lo han guo, stevioside, acesulfame, sucralose, and mixtures thereof. Even more preferred among these examples include erythritol, fructooligosaccharides, lo han guo, acesulfame, sucralose, and mixtures thereof. However, as stated below, in certain embodiments of the present invention it is not preferred to include aspartame in the present compositions.

Naturally occurring sweeteners or their purified extracts, such as stevioside, the protein sweetener thaumatin, lo han guo (disclosed in, for example, Fischer et al., U.S. Pat. No. 5,433,965, issued Jul. 18, 1995), and the like can be utilized as the sweetening agent herein.

The preferred fructooligosaccharides are a mixture of fructooligosaccharides composed of a chain of fructose molecules linked to a molecule of sucrose. Most preferably, these fructooligosaccharides have a nystose to kestose to fructosyl-nystose ratio of about 40:50:10, by weight of the composition. Preferred fructooligosaccharides may be obtained by enzymatic action of fructosyltransferase on sucrose such as those which are, for example, commercially available from Beghin-Meiji Industries, Neuilly-sur-Seine, France.

Other non-limiting examples of such sweetening agents include polyols, which are preferred due to their ability to provide the bulk of sugar, but without the calories and glycemic contribution of sugar. Thus, polyols are particularly useful for controlling increases in blood glucose and insulin levels. Non-limiting examples of well-known polyols for such use as sweetening agents include erythritol, mannitol, isomalt, lactitol, maltitol, sorbitol, and xylitol.

Erythritol is a particularly preferred sweetening agent for use herein. Erythritol is a polyol which is commonly used as a bulk sweetener in reduced calorie foods. Erythritol provides about 70% of the “sweetness” relative to sucrose, and about 5% of the calories relative to sucrose. In the United States, erythritol is typically labeled to provide approximately 0.2 calories per gram. Similarly, mannitol, isomalt, lactitol, maltitol, sorbitol, and/or xylitol may be utilized in the present compositions to provide the bulk of traditional sugars with exceedingly less caloric intake and blood sugar contribution.

Sucralose is also particularly preferred for use herein. Sucralose has little to no effect on sugar or carbohydrate metabolism, blood glucose elevation, or insulin production. Sucralose is commercially available from, for example, McNeil Specialty Products Company, New Brunswick, N.J.

Non-limiting examples of other non-caloric sweetening agents include aspartame, saccharin, cyclamates, acesulfame K, L-aspartyl-L-phenylalanine lower alkyl ester sweeteners, L-aspartyl-D-alanine amides such as, for example, those disclosed in Brennan et al., U.S. Pat. No. 4,411,925, issued 1983, L-aspartyl-D-serine amides such as, for example, those disclosed in Brennan et al., U.S. Pat. No. 4,399,163, issued 1983, L-aspartyl-hydroxymethyl alkane amide sweeteners such as, for example, those disclosed in Brand, U.S. Pat. No. 4,338,346, issued 1982, L-aspartyl-1-hydroxyethylalkane amide sweeteners such as, for example, those disclosed in Rizzi, U.S. Pat. No. 4,423,029, issued 1983, glycyrrhizins, and synthetic alkoxy aromatics.

However, in certain embodiments of the present invention aspartame is not preferred for use herein. Therefore, in these embodiments, the present compositions are substantially free of aspartame. As used herein, the term “substantially free” with respect to aspartame means that the compositions comprise less than about 0.5% of aspartame, preferably less than about 0.25%, more preferably less than about 0.1%, and most preferably less than about 0.01% of aspartame, all by weight of the composition.

The amount of the sweetening agent used in the compositions of the present invention typically depends upon the particular sweetening agent used and the sweetness intensity desired. Typically, the present compositions comprise from about 0.00001% to about 75% total sweetening agent, by weight of the composition. Dry beverage compositions (i.e., those which are suitable for dilution to provide a concentrate or ready-to-drink beverage composition) will typically comprise from about 0.0001% to about 75%, more preferably from about 5% to about 65%, even more preferably from about 10% to about 60%, and most preferably from about 20% to about 55% total sweetening agent, all by weight of the composition (i.e., the dry beverage composition). Concentrates which are suitable for dilution to provide a ready-to-drink beverage composition will typically comprise from about 0.0001% to about 75%, more preferably from about 1% to about 50%, even more preferably from about 2% to about 40%, and most preferably from about 5% to about 30% total sweetening agent, all by weight of the composition (i.e., the concentrate). Ready-to-drink beverage compositions will typically comprise from about 0.0001% to about 50%, more preferably from about 0.001% to about 25%, even more preferably from about 0.01% to about 10%, and most preferably from about 0.25% to about 5% total sweetening agent, all by weight of the composition (i.e., the ready-to-drink beverage composition). Wherein mixtures of sweetening agents are utilized, the relative weight percentages of each sweetening agents collectively provide the amount of total sweetening agent present in the composition.

### Optional Components of the Present Compositions

The compositions of the present invention may be utilized in food, beverage, pharmaceutical, over-the-counter, and dietary supplement compositions. Particularly preferred for use herein include syrups, concentrates suitable for dilution to provide a ready-to-drink composition, powders or other dry compositions suitable for dilution to provide a ready-to-drink composition, and ready-to-drink compositions.

Among these, ready-to-drink compositions are most preferred, with dry beverage compositions and concentrates also preferred. As referred to herein, dry beverage compositions will be suitable for dilution with water or other liquids to form a concentrate or ready-to-drink beverage composition. As defined herein, “dry” with reference to beverage compositions means that the composition comprises less than about 5% water, more preferably less than about 1% water, by weight of the dry beverage composition.

Food compositions are also useful herein. Preferred food compositions include chews, candies, gum, and other confectionery products, bars (including “health” bars and dessert bars), as well as other baked goods and spreads.

Tablets, capsules, pills, and other such forms are also useful herein.

Consistent with these various uses, the compositions of the present invention may comprise additional optional components to enhance, for example, their performance in providing joint health, bone health, other health benefits, a desirable nutritional profile, and/or organoleptic properties. For example, one or more caloric sweeteners, omega-3-fatty acids, bracers, flavanols, milk solids, soluble fibers, nutrients, flavoring agents, coloring agents, preservatives, acidulants, emulsifiers, thickeners, oils, water, carbonation components, and the like may be included in the compositions herein. Such optional components may be dispersed, solubilized, or otherwise mixed into the present compositions. These components may be added to the compositions herein provided they do not substantially hinder the properties of the composition, particularly the provision of joint and/or bone health. Non-limiting examples of optional components suitable for use herein are given below.

Caloric Sweeteners

As stated herein, a distinct advantage of the present invention is provision of compositions comprising a chondroprotective agent which avoid a high caloric intake and/or high glycemic index. However, this does not preclude the use of more traditional caloric sweeteners and, therefore, one or more caloric sweeteners such as glucose, dextrose, sucrose, and/or fructose may be utilized herein. For example, sucrose and/or fructose may be commonly used in combination with the requirements of the present invention.

Fructose can be obtained or provided as liquid fructose, high fructose corn syrup, dry fructose or fructose syrup, but is preferably provided as high fructose corn syrup. High fructose corn syrup (HFCS) is commercially available as HFCS-42, HFCS-55 and HFCS-90, which comprise 42%, 55% and 90%, respectively, by weight of the sugar solids therein, as fructose. Fructose is also found in fruit juices, and fruit juices are included herein as a preferred embodiment of the present invention.

Notwithstanding, it is preferred that these caloric sweeteners are minimized where practical, and the requirements of the present invention allow for this minimization while still providing a useful composition. Therefore, the present compositions may comprise a caloric sweetener selected from glucose, dextrose, sucrose, and fructose (more preferably selected from sucrose and fructose).

Omega-3-Fatty Acids

In a particularly preferred embodiment of the present invention, one or more omega-3-fatty acids may be added to the present compositions. Omega-3-fatty acids are anti-inflammatory compounds which act as competitive inhibitors of the arachidonic acid cascade. The omega-3-fatty acids are precursors to the synthesis of prostaglandins which function in mammals to regulate inflammation. See e.g., Burger, U.S. Pat. No. 5,843,919, issued Dec. 1, 1998.

The omega-3-fatty acid optionally utilized herein may be any omega-3-fatty acid or combination of omega-3-fatty acids. Non-limiting examples of omega-3-fatty acids which are suitable for use herein include eicosapentaenoic acid (also known as EPA), docosahexaenoic acid (also known as DHA), and mixtures thereof.

Optionally, the omega-3-fatty acid, as well as all other oil soluble components described herein, can be added to the present compositions via an emulsion and/or encapsulation. Additionally, in essentially dry compositions, the omega-3-fatty acid may be spray dried according to commonly known techniques.

Wherein one or more omega-3-fatty acids is utilized in the present compositions, the ratio of the first component herein and the omega-3-fatty acids is often important for optimization of health benefits, particularly joint health benefits, bone health benefits, and anti-inflammation. Preferably, the ratio of the first component to the total omega-3-fatty acid(s) present in the composition (on a weight to weight basis) is from about 95:5 to about 5:95, more preferably from about 75:25 to about 25:75, most preferably from about 60:40 to about 40:60. The dosage of omega-3-fatty acid(s) included in the composition is therefore preferably administered according to these guidelines. Typical dosage levels of the first component has been detailed herein above.

Bracers

As is commonly known in the art, bracers can be obtained by extraction from a natural source or can be synthetically produced. Non-limiting examples of bracers include methylxanthines, e.g., caffeine, theobromine, and theophylline. Additionally, numerous other xanthine derivatives have been isolated or synthesized, which may be utilized as a bracer in the compositions herein. See e.g., Bruns, Biochemical Pharmacology, Vol. 30, pp. 325-333 (1981) which describes, inter alia, xanthine, 9-methyl xanthine, 7-methyl xanthine, 3-methyl xanthine, 3,7-dimethyl xanthine, 8-chloromethyl-3,7-dimethyl xanthine, 8-hydroxymethyl-3,7-dimethyl xanthine, 3,7-diethyl xanthine, 3,7-bis-(2-hydroxyethyl) xanthine, 3-propyl-7-(dimethylaminoethyl) xanthine, 1-methyl xanthine, 1,9-dimethyl xanthine, 1-methyl-8-methylthio xanthine, 8-phenyl-1-methyl xanthine, 1,7-dimethyl xanthine, 1,7-dimethyl-8-oxo xanthine, 1,3-dimethyl xanthine, 1,3,9-trimethyl xanthine, 8-fluoro theophylline, 8-chloro theophylline, 8-bromo theophylline, 8-thio theophylline, 8-methylthio theophylline, 8-ethylthio theophylline, 8-nitro theophylline, 8-methylamino theophylline, 8-dimethylamino theophylline, 8-methyl theophylline, 8-ethyl theophylline, 8-propyl theophylline, 8-cyclopropyl theophylline, theophylline-8-propionate (ethyl ester), 8-benzyl theophylline, 8-cyclopentyl theophylline, 8-cyclohexyl theophylline, 8-(3-indolyl) theophylline, 8-phenyl theophylline, 9-methyl-8-phenyl theophylline, 8-(p-chlorophenyl) theophylline, 8-(p-bromophenyl) theophylline, 8-(p-methoxyphenyl) theophylline, 8-(p-nitrophenyl) theophylline, 8-(p-dimethylaminophenyl) theophylline, 8-(p-methylphenyl) theophylline, 8-(3,4-dichlorophenyl) theophylline, 8-(m-nitrophenyl) theophylline, 8-(o-nitrophenyl) theophylline, 8-(o-carboxyphenyl) theophylline, 8-(1-naphthyl) theophylline, 8-(2,6-dimethyl-4-hydroxyphenyl) theophylline, 7-methoxy-8-phenyl theophylline, 1,3,7-trimethyl xanthine, S-chloro caffeine, S-oxo caffeine, S-methoxy caffeine, S-methylamino caffeine, 8-diethylamino caffeine, 8-ethyl caffeine, 7-ethyl theophylline, 7-(2-chloroethyl) theophylline, 7-(2-hydroxyethyl) theophylline, 7-(carboxymethyl) theophylline, 7-(carboxymethyl) theophylline (ethyl ester), 7-(2-hydroxypropyl) theophylline, 7-(2,3-dihydroxypropyl) theophylline, 7-b-D-ribofuranosyl theophylline, 7-(glycero-pent-2-enopyranosyl) theophylline, 7-phenyl theophylline, 7,8-diphenyl theophylline, 1-methyl-3,7-diethyl xanthine, 1-methyl-3-isobutyl xanthine, 1-ethyl-3,7-dimethyl xanthine, 1,3-diethyl xanthine, 1,3,7-triethyl xanthine, 1-ethyl-3-propyl-7-butyl-8-methyl xanthine, 1,3-dipropyl xanthine, 1,3-diallyl xanthine, 1-butyl-3,7-dimethyl xanthine, 1-hexyl-3,7-dimethyl xanthine, and 1-(5-oxohexyl)-3,7-dimethyl xanthine.

Additionally, one or more of these bracers are present in, for example, coffee, tea, kola nut, cacao pod, mate', yaupon, guarana paste, and yoco. Natural plant extracts are the preferred sources of bracers as they may contain other compounds that delay the bioavailability of the bracer thus they may provide mental refreshment and alertness without tension or nervousness.

The most preferred methylxanthine is caffeine. Caffeine may be obtained from the aforementioned plants and their waste or, alternatively, may be synthetically prepared. Preferred botanical sources of caffeine which may be utilized as a complete or partial source of caffeine include green tea, guarana, mate', black tea, cola nuts, cocoa, and coffee. As used herein, green tea, guarana, coffee, and mate' are the most preferred botanical sources of caffeine, most preferably green tea, guarana, and coffee. Mate' may have the additional benefit of an appetite suppressing effect and may be included for this purpose as well. The total amount of caffeine, in any embodiment of the present invention, includes the amount of caffeine naturally present in the tea extract, flavoring agent, botanical and any other components, as well as any added caffeine.

Any bracer utilized herein is preferably present in physiologically relevant amounts, which means that the sources used in the practice of this invention provide a safe and effective quantity to achieve the desired mental alertness.

Wherein a bracer is utilized in the present compositions, such compositions will preferably comprise from about 0.0005% to about 1%, more preferably from about 0.003% to about 0.5%, still more preferably from about 0.003% to about 0.2%, even more preferably from about 0.005% to about 0.05%, and most preferably from about 0.005% to about 0.02% of a bracer, by weight of the composition. Of course, as the skilled artisan will comprehend, the actual amount of bracer added will depend its biological effect, for example, effect of mental alertness on the consumer.

Flavanols

Flavanols are natural substances present in a variety of plants (e.g., fruits, vegetables, and flowers). The flavanols which may be utilized in the present invention can be extracted from, for example, fruit, vegetables, green tea or other natural sources by any suitable method well known to those skilled in the art. For example, extraction with ethyl acetate or chlorinated organic solvents is a common method to isolate flavanols from green tea. Flavanols may be extracted from either a single plant or mixtures of plants. Many fruits, vegetables, and flowers contain flavanols but to a lesser degree relative to green tea. Plants containing flavanols are known to those skilled in the art. Examples of the most common flavanols which are extracted from tea plants and other members of the Catechu gambir (Uncaria family) include, for example, catechin, epicatechin, gallocatechin, epigallocatechin, epicatechin gallate, and epigallocatechin gallate.

The flavanols utilized in all compositions of the present invention can be in the form of a tea extract. The tea extract can be obtained from the extraction of unfermented teas, fermented teas, partially fermented teas, and mixtures thereof. Preferably, the tea extracts are obtained from the extraction of unfermented and partially fermented teas. The most preferred tea extracts are obtained from green tea. Both hot and cold extracts can be used in the present invention. Suitable methods for obtaining tea extracts are well known. See e.g., Ekanayake, U.S. Pat. No. 5,879,733, issued Mar. 9, 1999; Tsai, U.S. Pat. No. 4,935,256, issued June 1990; Lunder, U.S. Pat. No. 4,680,193, issued July 1987; and Creswick, U.S. Pat. No. 4,668,525, issued May 26, 1987.

The preferred source of flavanols in the compositions of the present invention is green tea. Wherein green tea, and in particular the flavanols present in green tea, are incorporated into the beverage, the present inventors have discovered that the flavanols are at least partially responsible for delaying the bioavailability of bracers, which contributes to the reduction and/or elimination of nervousness and tension typically associated with such bracers.

Alternatively, these same flavanols may be prepared by synthetic or other appropriate chemical methods and incorporated into the present compositions. Flavanols, including catechin, epicatechin, and their derivatives are commercially available.

The amount of flavanols in the compositions of the present invention can vary. However, wherein one or more flavanols are utilized, preferably from about 0.001% to about 5%, more preferably from about 0.001% to about 2%, even more preferably from about 0.01% to about 1%, and most preferably from about 0.01% to about 0.05% of one or more flavanols is utilized, by weight of the composition.

Milk Solids and Other Proteins

One or more milk solids may also optionally be included in the compositions of the present invention. As used herein, milk solids means milk from one or more mammals or a plant-derived milk, and includes, for example, fermented milk, lactic acid beverages obtained by lactic acid fermentation or otherwise acidified, sterilized milk base, liquid milk, and milk products such as skim milk powder or whole milk powder or other powdered forms of milk. As used herein, milk solids means the solids content or dry matter of milk base.

Wherein one or more milk solids is utilized, the desired total level of milk solids, calculated on a milk solids basis for the compositions of the present invention, is typically from about 0.001% to about 15%, preferably from about 0.005% to about 10%, and most preferably from about 0.1% to about 5%.

Other proteins, such as soy, whey, casseinates, including isolates of the foregoing, may be utilized in the present compositions. The level of each of these proteins will vary, and may be readily determined by the ordinarily skilled artisan.

Soluble Fibers

One or more soluble fibers may also optionally be included in the compositions of the present invention to provide, for example, nutritive benefits. Soluble fibers which can be used singularly or in combination in all embodiments of the present invention include but are not limited to pectins, psyllium, guar gum, xanthan gum, alginates, gum arabic, fructo-oligosaccharides, inulin, agar, and carrageenan. Preferred among these soluble fibers are at least one of guar gum, xanthan, and carrageenan, most preferably at least one of guar gum and xanthan. These soluble fibers may also serve as stabilizing agents in the various embodiments of this invention.

Particularly preferred soluble fibers for use herein are glucose polymers, preferably those which have branched chains. Preferred among these soluble fibers is one marketed under the trade name Fibersol2, commercially available from Matsutani Chemical Industry Co., Itami City, Hyogo, Japan.

Pectin and fructo-oligosaccharides are also preferred soluble fibers herein. Even more preferably, pectin and fructo-oligosaccharides are used in combination. The preferred ratio of pectin to fructo-oligosaccharide is from about 3:1 to about 1:3, by weight of the composition. The preferred pectins have a degree of esterification higher than about 65%.

The preferred fructo-oligosaccharides are a mixture of fructo-oligosaccharides composed of a chain of fructose molecules linked to a molecule of sucrose. Most preferably, they have a nystose to kestose to fructosyl-nystose ratio of about 40:50:10, by weight of the composition. Preferred fructo-oligosaccharides may be obtained by enzymatic action of fructosyltransferase on sucrose such as those which are, for example, commercially available from Beghin-Meiji Industries, Neuilly-sur-Seine, France.

Preferred pectins are obtained by hot acidic extraction from citrus peels and may be obtained, for example, from Danisco Co., Braband, Denmark.

Wherein a soluble fiber is utilized, the desired total level of soluble dietary fiber for the compositions of the present invention is from about 0.01% to about 15%, preferably from about 0.1% to about 5%, more preferably from about 0.1% to about 3%, and most preferably from about 0.2% to about 2%, by weight of the composition.

Nutrients

The compositions herein may optionally, but preferably, be fortified further with one or more nutrients, especially one or more vitamins and/or minerals. The U.S. Recommended Daily Intake (USRDI) for vitamins and minerals are defined and set forth in the Recommended Daily Dietary Allowance-Food and Nutrition Board, National Academy of Sciences-National Research Council.

Unless otherwise specified herein, wherein a given mineral is present in the composition, the composition typically comprises at least about 1%, preferably at least about 5%, more preferably from about 10% to about 200%, even more preferably from about 40% to about 150%, and most preferably from about 60% to about 125% of the USRDI of such mineral. Unless otherwise specified herein, wherein a given mineral is present in the composition, the composition comprises at least about 1%, preferably at least about 5%, more preferably from about 10% to about 200%, even more preferably from about 20% to about 150%, and most preferably from about 25% to about 120% of the USRDI of such vitamin.

Non-limiting examples of such further vitamins and minerals, include niacin, thiamin, folic acid, pantothenic acid, biotin, vitamin A, vitamin C, vitamin B₂, vitamin B₃, vitamin B₆, vitamin B₁₂, vitamin D, vitamin E, vitamin K, iron, zinc, copper, phosphorous, iodine, chromium, molybdenum, and fluoride. Preferably, wherein a further vitamin or mineral is utilized the vitamin or mineral is selected from niacin, thiamin, folic acid, iodine, vitamin A, vitamin C, vitamin B₆, vitamin B₁₂, vitamin D, vitamin E, iron, zinc, and calcium. Preferably, at least one vitamin is selected from vitamin C, vitamin B₆, vitamin B₁₂, vitamin E, pantothenic acid, niacin, and biotin. Also preferably, the composition comprises vitamin C and one or more other vitamins selected from vitamin B₆, vitamin B₁₂, vitamin E, pantothenic acid, niacin, and biotin.

Commercially available vitamin A sources may also be included in the present compositions. As used herein, “vitamin A” includes, but is not limited to, vitamin A (retinol), β-carotene, retinol palmitate, and retinol acetate. The vitamin A may be in any form, for example, an oil, beadlets, or encapsulated. Wherein vitamin A is present in the compositions herein, the composition comprises at least about 1%, preferably at least about 5%, more preferably from about 10% to about 200%, even more preferably from about 15% to about 150%, and most preferably from about 20% to about 120% of the USRDI of such vitamin. Wherein vitamin A is present in the compositions herein, it is especially preferred to include about 25% of the USRDI of vitamin A. The quantity of vitamin A to be added is dependent on processing conditions and the amount of vitamin A deliver desired after storage. Preferably, wherein vitamin A is included within the present compositions, the compositions comprise from about 0.0001% to about 0.2%, more preferably from about 0.0002% to about 0.12%, also preferably from about 0.0003% to about 0.1%, even more preferably from about 0.0005% to about 0.08%, and most preferably from about 0.001% to about 0.06% of vitamin A, by weight of the composition.

Commercially available sources of vitamin B₂ (also known as riboflavin) may be utilized in the present compositions. Wherein vitamin B₂ is present in the compositions herein, the composition comprises at least about 1%, preferably at least about 5%, more preferably from about 5% to about 200%, even more preferably from about 10% to about 150%, and most preferably from about 10% to about 120% of the USRDI of such vitamin. Wherein vitamin B₂ is present in the compositions herein, it is especially preferred to include from about 15% to about 35% of the USRDI of vitamin B₂.

Vitamin C (ascorbic acid) is a particularly preferred optional ingredient for use herein. Without intending to be limited by theory, it is believed that vitamin C may be utilized to enhance the benefits herein, by serving as a co-factor for the enzyme which cross-links collagen.

Encapsulated ascorbic acid and edible salts of ascorbic acid can also be used. Wherein vitamin C is present in the compositions herein, the composition comprises at least about 1%, preferably at least about 5%, more preferably from about 10% to about 200%, even more preferably from about 20% to about 150%, and most preferably from about 25% to about 120% of the USRDI of such vitamin. Wherein vitamin C is present in the compositions herein, it is especially preferred to include about 100% of the USRDI of vitamin C. The quantity of vitamin C to be added is dependent on processing conditions and the amount of vitamin C deliver desired after storage. Preferably, wherein vitamin C is included within the present compositions, the compositions comprise from about 0.005% to about 0.2%, more preferably from about 0.01% to about 0.12%, also preferably from about 0.02% to about 0.1%, even more preferably from about 0.02% to about 0.08%, and most preferably from about 0.03% to about 0.06% of vitamin C, by weight of the composition.

Nutritionally supplemental amounts of other vitamins which may be incorporated herein include, but are not limited to, vitamins B₆ and B₁₂, folic acid, niacin, pantothenic acid, folic acid, vitamin D, and vitamin E. Wherein the composition comprises one of these vitamins, the composition preferably comprises at least 5%, preferably at least 25%, and most preferably at least 35% of the USRDI for such vitamin.

Minerals which may optionally be included in the compositions herein are, for example, calcium, manganese, magnesium, boron, zinc, iodine, iron, and copper. Minerals may be, for example, salts, chelated, complexed, or in colloidal form.

Any soluble salt of these minerals suitable for inclusion edible compositions can be used, for example, magnesium citrate, magnesium gluconate, magnesium sulfate, zinc chloride, zinc sulfate, potassium iodide, copper sulfate, copper gluconate, and copper citrate.

Manganese is a particularly preferred mineral for use herein, as this mineral is involved in the synthesis of glycosaminoglycans, collagen, and glycoproteins. Additionally manganese deficiencies can lead to abnormal bone growth, inflamed joints, bone loss, and arthritis. Manganese ascorbate is a particularly preferred form of manganese for use herein. Typical manganese dosages range from about 0 mg to about 1000 mg, more preferably from about 50 mg to about 950 mg, and most preferably from about 50 mg to about 250 mg for a human or large mammal (e.g., horse).

Boron is a particularly preferred mineral for use herein, as this mineral is necessary for osteocalcin formation in bone.

Calcium is a particularly preferred mineral for use in the present invention. Preferred sources of calcium include, for example, amino acid chelated calcium, calcium carbonate, calcium oxide, calcium hydroxide, calcium sulfate, calcium chloride, calcium phosphate, calcium hydrogen phosphate, calcium dihydrogen phosphate, calcium citrate, calcium malate, calcium titrate, calcium gluconate, calcium realate, calcium tantrate, and calcium lactate, and in particular calcium citrate-malate. The form of calcium citrate-malate is described in, e.g., Mehansho et al., U.S. Pat. No. 5,670,344, issued Sep. 23, 1997; Diehl et al., U.S. Pat. No. 5,612,026, issued Mar. 18, 1997; Andon et al., U.S. Pat. No. 5,571,441, issued Nov. 5, 1996; Meyer et al., U.S. Pat. No. 5,474,793, issued Dec. 12, 1995; Andon et al., U.S. Pat. No. 5,468,506, issued Nov. 21, 1995; Burkes et al., U.S. Pat. No. 5,445,837, issued Aug. 29, 1995; Dake et al., U.S. Pat. No. 5,424,082, issued Jun. 13, 1995; Burkes et al., U.S. Pat. No. 5,422,128, issued Jun. 6, 1995; Burkes et al., U.S. Pat. No. 5,401,524, issued Mar. 28, 1995; Zuniga et al., U.S. Pat. No. 5,389,387, issued Feb. 14, 1995; Jacobs, U.S. Pat. No. 5,314,919, issued May 24, 1994; Saltman et al., U.S. Pat. No. 5,232,709, issued Aug. 3, 1993; Camden et al., U.S. Pat. No. 5,225,221, issued Jul. 6, 1993; Fox et al., U.S. Pat. No. 5,215,769, issued Jun. 1, 1993; Fox et al., U.S. Pat. No. 5,186,965, issued Feb. 16, 1993; Saltman et al., U.S. Pat. No. 5,151,274, issued Sep. 29, 1992; Kochanowski, U.S. Pat. No. 5,128,374, issued Jul. 7, 1992; Mehansho et al., U.S. Pat. No. 5,118,513, issued Jun. 2, 1992; Andon et al., U.S. Pat. No. 5,108,761, issued Apr. 28, 1992; Mehansho et al., U.S. Pat. No. 4,994,283, issued Feb. 19, 1991; Nakel et al., U.S. Pat. No. 4,786,510, issued Nov. 22, 1988; and Nakel et al., U.S. Pat. No. 4,737,375, issued Apr. 12, 1988. Preferred compositions of the present invention will comprise from about 0.01% to about 0.5%, more preferably from about 0.03% to about 0.2%, even more preferably from about 0.05% to about 0.15%, and most preferably from about 0.1% to about 0.15% of calcium, by weight of the composition.

Iron may also be utilized in the compositions and methods of the present invention. Acceptable forms of iron are well-known in the art. The amount of iron compound incorporated into the composition will vary widely depending upon the level of supplementation desired in the final composition and the targeted consumer. Iron fortified compositions of the present invention typically contain from about 5% to about 100%, preferably from about 15% to about 50%, and most preferably about 20% to about 40% of the USRDI for iron.

Ferrous iron is typically better utilized by the body than ferric iron. Highly bioavailable ferrous salts that can be used in the ingestible compositions of the present invention are ferrous sulfate, ferrous fumarate, ferrous succinate, ferrous gluconate, ferrous lactate, ferrous tartarate, ferrous citrate, ferrous amino acid chelates, as well as mixtures of these ferrous salts. While ferrous iron is typically more bioavailable, certain ferric salts can also provide highly bioavailable sources of iron. Highly bioavailable ferric salts that can be used in the food or beverage compositions of the present invention are ferric saccharate, ferric ammonium citrate, ferric citrate, ferric sulfate, as well as mixtures of these ferric salts. Combinations or mixtures of highly bioavailable ferrous and ferric salts can be used in these edible mixes and ready-to-serve beverages. The preferred sources of highly bioavailable iron are ferrous fumarate and ferrous amino acid chelates.

Ferrous amino acid chelates particularly suitable as highly bioavailable iron sources for use in the present invention are those having a ligand to metal ratio of at least 2:1. For example, suitable ferrous amino acid chelates having a ligand to metal mole ratio of two are those of formula:

Fe(L)₂

where L is an alpha amino acid, dipeptide, tripeptide, or quadrapeptide ligand. Thus, L can be any ligand which is a naturally occurring alpha amino acid selected from alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, and valine; or dipeptides, tripeptides, or quadrapeptides formed by any combination of these alpha amino acids. See e.g., Ashmead et al., U.S. Pat. No. 4,863,898, issued Sep. 5, 1989; Ashmead, U.S. Pat. No. 4,830,716, issued May 16, 1989; and Ashmead, U.S. Pat. No. 4,599,152, issued Jul. 8, 1986, all of which are incorporated by reference. Particularly preferred ferrous amino acid chelates are those where the reacting ligands are glycine, lysine, and leucine. Most preferred is the ferrous amino acid chelate sold under the mark FERROCHEL (Albion Laboratories, Salt Lake City, Utah) wherein the ligand is glycine.

In addition to these highly bioavailable ferrous and ferric salts, other sources of bioavailable iron can be included in the food and beverage compositions of the present invention. Other sources of iron particularly suitable for fortifying compositions of the present invention included certain iron-sugar-carboxylate complexes. In these iron-sugar-carboxylate complexes, the carboxylate provides the counterion for the ferrous (preferred) or ferric iron. The overall synthesis of these iron-sugar-carboxylate complexes involves the formation of a calcium-sugar moiety in aqueous media (for example, by reacting calcium hydroxide with a sugar, reacting the iron source (such as ferrous ammonium sulfate) with the calcium-sugar moiety in aqueous media to provide an iron-sugar moiety, and neutralizing the reaction system with a carboxylic acid (the “carboxylate counterion”) to provide the desired iron-sugar-carboxylate complex. Sugars that can be used to prepare the calcium-sugar moiety include any of the ingestible saccharidic materials, and mixtures thereof, such as glucose, sucrose and fructose, mannose, galactose, lactose, maltose, and the like, with sucrose and fructose being the more preferred. The carboxylic acid providing the “carboxylate counterion” can be any ingestible carboxylic acid such as citric acid, malic acid tartaric acid, lactic acid, succinic acid, propionic acid, etc., as well as mixtures of these acids.

These iron-sugar-carboxylate complexes can be prepared in the manner described in, e.g., Nakel et al., U.S. Pat. Nos. 4,786,510 and 4,786,518, issued Nov. 22, 1988, both of which are incorporated by reference. These materials are referred to as “complexes”, but they may exist in solution as complicated, highly hydrated, protected colloids; the term “complex” is used for the purpose of simplicity.

Zinc may also be utilized in the compositions and methods of the present invention. Acceptable forms of zinc are well-known in the art. Zinc fortified compositions of the present invention typically contain from about 5% to about 100%, preferably from about 15% to about 50%, and most preferably about 25% to about 45% of the USRDI for zinc. The zinc compounds which can be used in the present invention can be in any of the commonly used forms such as, e.g., zinc sulfate, zinc chloride, zinc acetate, zinc gluconate, zinc ascorbate, zinc citrate, zinc aspartate, zinc picolinate, amino acid chelated zinc, and zinc oxide. Zinc gluconate and amino acid chelated zinc are particularly preferred.

Flavoring Agents

One or more flavoring agents are recommended for the embodiments of the present invention in order to enhance their palatability. Any natural or synthetic flavor agent can be used in the present invention. For example, it is highly preferred to include fruit juice in the present compositions. It is also a preferred embodiment to include one or more botanical and/or fruit flavors may be utilized herein. Thus, the flavor agent can also comprise a blend of various flavors. As used herein, such flavors may be synthetic or natural flavors.

Any of a variety of fruit juices and/or fruit juice concentrates may be incorporated herein including, for example, apple, strawberry, lemon, grapefruit, kiwi, lime, grape, tangerine, orange, cherry, raspberry, cranberry, peach, watermelon, passion fruit, pineapple, mango, cupuacu, guava, cocoa, papaya, and apricot fruit juices, as well as mixtures thereof (e.g., cranberry and apple), may be used. In a particularly preferred embodiment, the present compositions comprise greater than 0%, more preferably at least about 5%, still more preferably from about 5% to about 60%, even more preferably from about 5% to about 40%, and most preferably from about 5% to about 30% fruit juice, all by weight of the composition.

Fruit flavors may also be used. Particularly preferred fruit flavors are apple, strawberry, lemon, grapefruit, kiwi, lime, grape, tangerine, orange, cherry, raspberry, cranberry, peach, watermelon, and the like, as well as mixtures thereof. Blends of flavors (for example, tangerine-orange) are most preferred. Exotic and lactonic flavors such as, for example, passion fruit, pineapple, mango, cupuacu, guava, cocoa, papaya, and apricot, as well as mixtures thereof, may also be utilized. These fruit flavors can be derived from natural sources such as fruit juices and flavor oils, or may alternatively be synthetically prepared.

Preferred botanical flavors include, for example, tea (preferably black and green tea, most preferably green tea), aloe vera, guarana, ginseng, ginkgo, hawthorn, hibiscus, rose hips, chamomile, peppermint, fennel, ginger, licorice, lotus seed, schizandra, saw palmetto, sarsaparilla, safflower, St. John's Wort, curcuma, cardimom, nutmeg, cassia bark, buchu, cinnamon, jasmine, haw, chrysanthemum, water chestnut, sugar cane, lychee, bamboo shoots, vanilla, coffee, and the like. Preferred among these is tea, guarana, ginseng, ginko, and coffee. In particular, the combination of tea flavors, preferably green tea or black tea flavors (preferably green tea), optionally together with fruit flavors has an appealing taste. In another preferred embodiment, coffee is included within the present compositions. A combination of green tea and coffee in the present compositions is often preferred.

If desired, the flavor in the flavoring agent may be formed into emulsion droplets which are then dispersed in the beverage composition or concentrate. Because these droplets usually have a specific gravity less than that of water and would therefore form a separate phase, weighting agents (which can also act as clouding agents) can be used to keep the emulsion droplets dispersed in the beverage composition or concentrate. Examples of such weighting agents are brominated vegetable oils (BVO) and resin esters, in particular the ester gums. See L. F. Green, Developments in Soft Drinks Technology, Vol. 1, Applied Science Publishers Ltd., pp. 87-93 (1978) for a further description of the use of weighting and clouding agents in liquid beverages. Typically the flavoring agents are conventionally available as concentrates or extracts or in the form of synthetically produced flavoring esters, alcohols, aldehydes, terpenes, sesquiterpenes, and the like.

Coloring Agent

Small amounts of one or more coloring agents may be utilized in the compositions of the present invention. FD&C dyes (e.g., yellow #5, blue #2, red # 40) and/or FD&C lakes are preferably used. By adding the lakes to the other powdered ingredients, all the particles, in particular the colored iron compound, are completely and uniformly colored and a uniformly colored beverage mix is attained. Preferred lake dyes which may be used in the present invention are the FDA-approved Lake, such as Lake red #40, yellow #6, blue #1, and the like. Additionally, a mixture of FD&C dyes or a FD&C lake dye in combination with other conventional food and food colorants may be used. Riboflavin and b-carotene may also be used. Additionally, other natural coloring agents may be utilized including, for example, fruit, vegetable, and/or plant extracts such as grape, black currant, aronia, carrot, beetroot, red cabbage, and hibiscus.

The amount of coloring agent used will vary, depending on the agents used and the intensity desired in the finished composition. The amount can be readily determined by one skilled in the art. Generally, if utilized, the coloring agent should be present at a level of from about 0.0001% to about 0.5%, preferably from about 0.001% to about 0.1%, and most preferably from about 0.004% to about 0.1%, by weight of the composition.

Preservatives

Optionally, one or more preservatives may additionally be utilized herein. Preferred preservatives include, for example, sorbate, benzoate, and polyphosphate preservatives.

Preferably, wherein a preservative is utilized herein, one or more sorbate or benzoate preservatives (or mixtures thereof) are utilized. Sorbate and benzoate preservatives suitable for use in the present invention include sorbic acid, benzoic acid, and salts thereof, including (but not limited to) calcium sorbate, sodium sorbate, potassium sorbate, calcium benzoate, sodium benzoate, potassium benzoate, and mixtures thereof. Sorbate preservatives are particularly preferred. Potassium sorbate is particularly preferred for use in the present invention.

Wherein a composition comprises a preservative, the preservative is preferably included at levels from about 0.0005% to about 0.5%, more preferably from about 0.001% to about 0.4% of the preservative, still more preferably from about 0.001% to about 0.1%, even more preferably from about 0.001% to about 0.05%, and most preferably from about 0.003% to about 0.03% of the preservative, by weight of the composition. Wherein the composition comprises a mixture of one or more preservatives, the total concentration of such preservatives is preferably maintained within these ranges.

Acidulants

If desired, the present compositions may optionally comprise one or more acidulants. An amount of an acidulant may be used to maintain the pH of the composition. Compositions of the present invention preferably have a pH of from about 2 to about 10, more preferably from about 2 to about 7, still more preferably from about 2 to about 5, even more preferably from about 3 to about 5, and most preferably from about 3.5 to about 4.5. Beverage acidity can be adjusted to and maintained within the requisite range by known and conventional methods, e.g., the use of one or more of the aforementioned acidulants. Typically, acidity within the above recited ranges is a balance between maximum acidity for microbial inhibition and optimum acidity for the desired beverage flavor.

Organic as well as inorganic edible acids may be used to adjust the pH of the beverage, and may be added additional to the acid serving as part of the second component herein. The acids can be present in their undissociated form or, alternatively, as their respective salts, for example, potassium or sodium hydrogen phosphate, potassium or sodium dihydrogen phosphate salts. The preferred acids are edible organic acids which include citric acid, malic acid, fumaric acid, adipic acid, phosphoric acid, gluconic acid, tartaric acid, ascorbic acid, acetic acid, phosphoric acid or mixtures thereof. The most preferred acids are citric and malic acids.

The acidulant can also serve as an antioxidant to stabilize beverage components. Examples of commonly used antioxidant include but are not limited to ascorbic acid, EDTA (ethylenediaminetetraacetic acid), and salts thereof.

Emulsifiers and Oils

One or more emulsifiers and/or oils may also be included in the present compositions for texture and opacity purposes. Typical emulsifiers and oils useful herein include, for example, mono-di glycerides, lecithin, pulp, cotton seed oil, and vegetable oil.

Thickeners

One or more thickeners may be optionally added to the present compositions to, for example, provide control of viscosity and/or texture. Various thickeners are well-known in the art. Non-limiting examples of thickeners include cellulose compounds, gum ghatti, modified gum ghatti, xanthan gum, tragacanth gum, guar gum, gellan gum, locust bean gum, pectin, and mixtures thereof. See e.g., Kupper et al., U.S. Pat. No. 4,705,691, issued Nov. 10, 1987. Particularly preferred for use herein include xanthan gum, gellan gum, guar gum, and cellulose compounds.

Cellulose compounds are widely known in the art. Cellulose compounds are typically anionic polymers derived from cellulose. Non-limiting examples of cellulose compounds utilized herein include carboxymethylcellulose, methylcellulose, and hydroxyethylcellulose, hydroxypropylcellulose. The most preferred cellulose compound for use in the present compositions is carboxymethylcellulose, particularly sodium carboxymethylcellulose. Non-limiting examples of cellulose compounds include sodium carboxymethylcellulose (commercially available as Aqualon® 7HOF from Hercules, Inc., Wilmington, Del.

When present, the thickener is typically utilized in the present compositions at levels preferably from about 0.00001% to about 10%, more preferably from about 0.00001% to about 5%, still more preferably from about 0.00001% to about 1%, even more preferably from about 0.01% to about 0.2%, and most preferably from about 0.02% to about 0.05%, by weight of the composition.

Water

Water may be, and preferably is, included in the present compositions. Water is most preferably included in the beverage composition embodiments of the present invention. Wherein water is included within the compositions herein, the compositions preferably comprise at least about 10% water, more preferably at least about 20% water, still more preferably at least about 40% water, even more preferably at least about 75% water, and most preferably at least about 80% water. Still further, ready-to-drink beverage compositions will typically comprise from at least about 80% water to about 99.9% water. Beverage concentrates will typically comprise from at least about 10% water, more preferably at least about 20% water, and most preferably at least about 25% water. As defined herein, “dry” with reference to beverage compositions means that the composition comprises less than about 5% water, more preferably less than about 1% water, by weight of the composition. As used herein, the water of the composition includes all added water and any water present in combination components, for example, fruit juice.

Carbonation Component

Carbon dioxide can be introduced into the water which is mixed with a beverage concentrate or into the beverage composition after dilution to achieve carbonation. The carbonated beverage can be placed into a container, such as a bottle or can, and then sealed. Any conventional carbonation methodology may be utilized to make carbonated beverage compositions of this invention. The amount of carbon dioxide introduced into the beverage will depend upon the particular flavor system utilized and the amount of carbonation desired.

### Methods of Making

The present compositions are made according to methods which will be well known by the ordinarily skilled artisan. To illustrate, the compositions of the present invention may be prepared by dissolving, dispersing, or otherwise mixing all components singularly or in suitable combinations together and in water where appropriate, agitating with a mechanical stirrer until all of the ingredients have been solubilized or adequately dispersed. Where appropriate, all separate solutions and dispersed may then be combined. When using tea extracts which typically are pH sensitive, it is important to adjust the desired pH with an acidulant and/or buffer system before adding the tea extracts to the mixture. Wherein a shelf stable composition is desired, the final mixture can optionally, but preferably, be pasteurized or filled aseptically at appropriate process conditions.

In making a beverage composition, a beverage concentrate may optionally be formed first. One method to prepare the concentrate form of the beverage composition would be to start with less than the required volume of water that is used in the preparation of the beverage composition. Another method would be to partially dehydrate the finally prepared beverage compositions to remove only a portion of the water and any other volatile liquids present. Dehydration may be accomplished in accordance with well known procedures, such as evaporation under vacuum. The concentrate can be in the form of a relatively thick liquid. A syrup is typically formed by adding suitable ingredients such as electrolytes or emulsions to the beverage concentrate. The syrup is then mixed with water to form a finished beverage or finished beverage concentrate. The weight ratio of water to syrup is typically from about 1:1 to about 5:1.

Carbon dioxide can be introduced either into the water to be mixed with the beverage concentrate, or into the drinkable beverage composition, to achieve carbonation. The carbonated beverage composition can then be stored in a suitable container and then sealed. Techniques for making and carbonating beverage embodiments of the present invention are described in the following references: L. F. Green (ed.), Developments in Soft Drinks Technology, Vol. 1 (Elsevier, 1978); G. S. Cattell and P. M. Davies “Preparation and Processing of Fruit Juices, Cordials and Drinks”, Journal of the Society of Dairy Technology; Vol. 38 (1), pp. 21-27, A. H. Varnam and J. P. Sutherland, Beverages—Technology, Chemistry and Microbiology, Chapman Hall, 1994; and A. J. Mitchell (ed.), Formulation and Production of Carbonated Soft Drinks, Blackie and Sons Ltd., 1990.

Dry compositions, or essentially dry compositions of the present invention can be prepared by blending the proper amounts and ratios of all the required dry ingredients together. Alternatively, the finally prepared beverage compositions can be dehydrated to give a dry beverage composition of the present invention. The dry beverage composition, either as, for example, a powder, granules or tablets, can later be dissolved in a proper amount of water or other liquid, carbonated or non-carbonated, to make the beverage concentrate or ready-to-drink beverage composition. Alternatively, dry forms of the present invention may be incorporated in other compositions, including but not limited to cereal bars, breakfast bars, energy bars, and nutritional bars.

Other essentially dry compositions include, for example, tablets, capsules, granules, and dry powders. Tablets may contain suitable binders, lubricants, diluents, disintegrating agents, coloring agents, flavoring agents, flow-inducing agents, and melting agents. Suitable carriers and excipients that may be used to formulate dry forms of the present invention are described in, for example, Rober, U.S. Pat. No. 3,903,297, issued Sep. 2, 1975. Techniques and compositions for making dry forms useful in the methods of this invention are described in the following references: H. W. Houghton (ed.), Developments in Soft Drinks Technology, Vol. 3, Chapter 6, (Elsevier, 1984); Modern Pharmaceutics, Chapters 9 and 10 (Banker & Rodes (ed.), 1979); Liberman et al., Pharmaceutical Dosage Forms: Tablets (1981); and Ansel, Introduction to Pharmaceutical Dosage Forms, 2nd Ed., (1976).

### Kits of the Present Invention

The compositions of the present invention may be utilized in kits as described herein. The kits of the present invention comprise one or more compositions of the present invention together with information which informs a user of the kit, by words, pictures, and/or the like, that use of the kit will provide one or more general health and/or general physiological benefits including, but not limited to, joint health benefits (including relief from, prevention of, and/or inhibition of, arthritis and/or osteoarthritis, as well as enhanced flexibility), bone health benefits (including maintaining and/or building bones), anti-inflammation (e.g., pain relief). Additionally or alternatively, the kits may comprise one or more compositions of the present invention together with information which informs a user of the kit, by words, pictures, and/or the like, that use of the kit is suitable and/or beneficial for use by diabetic mammals (i.e., utility for diabetic mammals).

In a particularly preferred embodiment, the information is printed on a containment device directly or indirectly containing the composition, e.g., a bottle. As an example, these preferred kits may be in the form of one bottle containing the composition, or may be obtained as a plurality of bottles each containing the composition. For example, the kits may be obtained as one bottle, or cases of four, six, seven (e.g., a weekly supply), or eight bottles co-packaged together. Additionally, monthly kits may be obtained as cases of, for example, twenty-eight or thirty bottles co-packaged together. In this instance, as a non-limiting example, the information may be printed on each individual bottle and/or on a containment device containing the bottles.

As used herein, the information may be conveyed through words, pictures, symbols, and/or other visible descriptors. Such information need not utilize the actual words used herein, for example, “joint”, “bone”, “human”, “mammal”, or “diabetes” but rather use of words, pictures, symbols, and the like conveying the same or similar meaning are contemplated within the scope of this invention.

### Methods of the Present Invention

The methods of the present invention comprise orally administering (i.e., through ingestion) a composition of the present invention to a mammal, preferably a human, to treat joint dysfunction, bone dysfunction, and/or inflammation. The compositions are preferably ingested by mammals who experience joint and/or bone dysfunction or those who desire to maintain current joint and/or bone function (i.e., prophylactic use). Additionally or alternatively, the compositions may be suitable and/or beneficial for use by diabetic mammals by virtue of the inclusion of the sweetening agent herein. The compositions of this invention may also be ingested as a supplement to normal dietetic requirements. Frequency of administration is not limited, however, such administration is typically at least once weekly, more preferably at least 3 times weekly, and most preferably at least once daily. Administration will typically be ongoing.

As used herein, the term “orally administering” with respect to the mammal (preferably, human) means that the mammal ingests or is directed to ingest one or more compositions of the present invention for one or more of the purposes described herein, including treating joint dysfunction, bone dysfunction, and/or inflammation. For example, such direction may be oral direction (e.g., through oral instruction from, for example, a physician, health professional, sales professional or organization, and/or radio or television media (i.e., advertisement) or written direction (e.g., through written direction from, for example, a physician or other health professional (e.g., scripts), sales professional or organization (e.g., through, for example, marketing brochures, pamphlets, or other instructive paraphernalia), written media (e.g., internet, electronic mail, or other computer-related media), and/or packaging associated with the composition (e.g., a label present on a package containing the composition). As used herein, “written” means through words, pictures, symbols, and/or other visible descriptors. Such direction need not utilize the actual words used herein, for example, “joint”, “bone”, “inflammation”, “human”, or “mammal”, but rather use of words, pictures, symbols, and the like conveying the same or similar meaning are contemplated within the scope of this invention.

### EXAMPLES

The following are non-limiting examples of the present compositions which are prepared utilizing conventional methods. The following examples are provided to illustrate the invention and are not intended to limit the scope thereof in any manner.

### Example 1

A low-calorie dry beverage composition suitable for dilution to provide a ready-to-drink composition is prepared having the following components. About 10 grams of the dry composition is packaged in a single-serving pouch, and then co-packaged in a kit comprising 7 single-serving pouches (for example, a weekly dose) which is transportable and convenient for use by the user. The dry beverage composition may be diluted with water by the user to provide a 230 milliliter ready-to-drink beverage composition.

Two of the kits are obtained by a 50-year-old female diabetic human, intended as a two week dosage. The female dilutes the contents of one pouch once daily with water, and ingests the resulting ready-to-drink beverage composition. After a two week period, the female reports symptomatic pain relief and is able to perform various physical tasks with increased ease. Additionally, the female is able to ingest the composition without any appreciable increases in her blood glucose levels.

| Component | Wt % |
|---|---|
| Glucosamine HCl | 17.75 |
| Citric acid | 14.98 |
| Malic acid | 5.29 |
| Calcium hydroxide | 5.85 |
| Ascorbic acid | 0.81 |
| Color | 0.2 |
| Flavor | 7.1 |
| Xanthan gum | 0.49 |
| Crystalline sucralose | 0.18 |
| Erythritol | 47.34 |

### Example 2

A low-calorie ready-to-drink beverage composition, having about 16 grams of total carbohydrate per every 230 milliliters of the composition, is prepared by combining the following components in a conventional manner:

| Component | Wt % |
|---|---|
| Ascorbic acid | 0.07 |
| Calcium disodium EDTA | 0.003 |
| Calcium hydroxide | 0.25 |
| Citric acid | 0.63 |
| Erythritol | 2.0 |
| Crystalline fructose | 2.0 |
| Glucosamine HCl | 0.75 |
| Malic acid | 0.22 |
| Sodium benzoate | 0.002 |
| Sodium carboxymethylcellulose | 0.03 |
| Sucralose (25%) | 0.03 |
| Xanthan gum | 0.006 |
| Juice concentrates | 2.0 |
| Colors | 0.007 |
| Flavor oils | 0.04 |
| Water | quantum satis |

Various flavors of the beverage composition may be formulated according to standard techniques, for example, orange, grapefruit, cranberry, and/or cranberry-apple flavors.

In a preferred embodiment of the present invention, 28 PET bottles each containing 8 ounces of the composition are co-packaged in a convenient container (e.g., a box). This may be used as a monthly dose of the present composition.

The kit is obtained by a 60-year-old male diabetic human, intended as a monthly dosage. The male ingests the contents of one bottle daily, for twenty-eight days. About ten days into this period, the is able walk one mile daily without pain in his knees. Additionally, the male is able to ingest the composition without any appreciable increases in his blood glucose levels, and maintains his body mass during this period.

What is claimed is:

1. A composition comprising:
a) a chondroprotective agent selected from the group consisting of gelatin, cartilage, aminosugars, glycosaminoglycans, methylsulfonylmethane, precursors of methylsulfonylmethane, S-adenosylmethionine, and mixtures thereof;
b) a sweetening agent other than glucose, dextrose, sucrose, and fructose; and
c) at least about 10% water, by weight of the composition.

2. A composition according to claim 1 wherein the chondroprotective agent is selected from the group consisting of aminosugars, glycosaminoglycans, S-adenosylmethionine, and mixtures thereof.

3. A composition according to claim 2 wherein the sweetening agent is selected from the group consisting of sorbitol, mannitol, xylitol, erythritol, malitol, maltose, lactose, fructooligosaccharides, lo han guo, stevioside, acesulfame, aspartame, sucralose, saccharin, xylose, arabinose, levulose, isomalt, and ribose.

4. A composition according to claim 3 wherein the chondroprotective agent is selected from the group consisting of aminosugars, glycosaminoglycans, and mixtures thereof and wherein:
a) the aminosugars are selected from the group consisting of glucosamine and salts thereof; and
b) the glycosaminoglycans are selected from the group consisting of chondroitin and salts thereof.

5. A composition according to claim 4 further comprising at least one sweetener selected from the group consisting of sucrose, fructose, and mixtures thereof.

6. A composition according to claim 5 further comprising a caloric sweetener selected from the group consisting of glucose, dextrose, sucrose, fructose, and mixtures thereof.

7. A composition according to claim 4 wherein the sweetening agent is selected from the group consisting of xylitol, erythritol, fructooligosaccharides, lo han guo, stevioside, acesulfame, aspartame, sucralose, and mixtures thereof.

8. A composition according to claim 7 wherein the sweetening agent is selected from the group consisting of erythritol, sucralose, and mixtures thereof.

9. A composition according to claim 8 further comprising one or more beverage components selected from the group consisting of fruit juice, tea, milk solids, and mixtures thereof.

10. A composition according to claim 9 comprising at least about 75% water, by weight of the composition.

11. A composition according to claim 10 further comprising one or more nutrients.

12. A composition according to claim 10 further comprising one or more omega-3-fatty acids.

13. A composition comprising:
a) a chondroprotective agent selected from the group consisting of gelatin, cartilage, aminosugars, glycosaminoglycans, methylsulfonylmethane, precursors of methylsulfonylmethane, S-adenosylmethionine, salts thereof, and mixtures thereof; and
b) a sweetening agent other than glucose, dextrose, sucrose, and fructose;
wherein the composition is substantially free of aspartame.

14. A composition according to claim 13 wherein the chondroprotective agent is selected from the group consisting of aminosugars, glycosaminoglycans, S-adenosylmethionine, and mixtures thereof.

15. A composition according to claim 14 wherein the sweetening agent is selected from the group consisting of sorbitol, mannitol, xylitol, erythritol, malitol, maltose, lactose, fructooligosaccharides, lo han guo, stevioside, acesulfame, aspartame, sucralose, saccharin, xylose, arabinose, levulose, isomalt, and ribose.

16. A composition according to claim 15 wherein the chondroprotective agent is selected from the group consisting of aminosugars, glycosaminoglycans, and mixtures thereof and wherein:
a) the aminosugars are selected from the group consisting of glucosamine and salts thereof; and
b) the glycosaminoglycans are selected from the group consisting of chondroitin and salts thereof.

17. A composition according to claim 16 further comprising at least one sweetener selected from the group consisting of sucrose, fructose, and mixtures thereof.

18. A composition according to claim 17 further comprising a caloric sweetener selected from the group consisting of glucose, dextrose, sucrose, fructose, and mixtures thereof.

19. A composition according to claim 16 wherein the sweetening agent is selected from the group consisting of xylitol, erythritol, fructooligosaccharides, lo han guo, stevioside, acesulfame, aspartame, sucralose, and mixtures thereof.

20. A composition according to claim 19 wherein the sweetening agent is selected from the group consisting of erythritol, sucralose, and mixtures thereof.

21. A composition according to claim 20 further comprising one or more beverage components selected from the group consisting of fruit juice, tea, milk solids, and mixtures thereof.

22. A composition comprising:
a) a chondroprotective agent selected from the group consisting of gelatin, cartilage, aminosugars, glycosaminoglycans, methylsulfonylmethane, precursors of methylsulfonylmethane, S-adenosylmethionine, salts thereof, and mixtures thereof;
b) at least about 10% water, by weight of the composition; and
c) less than about 19 grams total carbohydrate per every 230 milliliters of the composition.

23. A composition according to claim 22 wherein the chondroprotective agent is selected from the group consisting of aminosugars, glycosaminoglycans, S-adenosylmethionine, and mixtures thereof.

24. A composition according to claim 23 comprising a sweetening agent selected from the group consisting of sorbitol, mannitol, xylitol, erythritol, malitol, maltose, lactose, fructooligosaccharides, lo han guo, stevioside, acesulfame, aspartame, sucralose, saccharin, xylose, arabinose, levulose, isomalt, and ribose.

25. A composition according to claim 24 wherein the chondroprotective agent is selected from the group consisting of aminosugars, glycosaminoglycans, and mixtures thereof and wherein:
a) the aminosugars are selected from the group consisting of glucosamine and salts thereof; and
b) the glycosaminoglycans are selected from the group consisting of chondroitin and salts thereof.

26. A composition according to claim 25 further comprising at least one sweetener selected from the group consisting of sucrose, fructose, and mixtures thereof.

27. A composition according to claim 26 further comprising a caloric sweetener selected from the group consisting of glucose, dextrose, sucrose, fructose, and mixtures thereof.

28. A composition according to claim 27 comprising less than about 18.5 grams total carbohydrate per every 230 milliliters of the composition.

29. A composition according to claim 28 further comprising one or more beverage components selected from the group consisting of fruit juice, tea, milk solids, and mixtures thereof.

30. A composition according to claim 28 comprising less than about 18 grams total carbohydrate per every 230 milliliters of the composition.

31. A composition according to claim 30 comprising at least about 75% water, by weight of the composition.

32. A composition according to claim 31 further comprising one or more nutrients.

33. A composition according to claim 31 further comprising one or more omega-3-fatty acids.

34. A composition comprising:
a) a chondroprotective agent selected from the group consisting of gelatin, cartilage, aminosugars, glycosaminoglycans, methylsulfonylmethane, precursors of methylsulfonylmethane, S-adenosylmethionine, salts thereof, and mixtures thereof; and
b) less than about 18 grams total carbohydrate per every 230 milliliters of the composition;
wherein the composition is substantially free of aspartame.

35. A composition according to claim 34 wherein the chondroprotective agent is selected from the group consisting of aminosugars, glycosaminoglycans, S-adenosylmethionine, and mixtures thereof.

36. A composition according to claim 35 comprising a sweetening agent selected from the group consisting of sorbitol, mannitol, xylitol, erythritol, malitol, maltose, lactose, fructooligosaccharides, lo han guo, stevioside, acesulfame, aspartame, sucralose, saccharin, xylose, arabinose, levulose, isomalt, and ribose.

37. A composition according to claim 36 wherein the chondroprotective agent is selected from the group consisting of aminosugars, glycosaminoglycans, and mixtures thereof and wherein:
a) the aminosugars are selected from the group consisting of glucosamine and salts thereof; and
b) the glycosaminoglycans are selected from the group consisting of chondroitin and salts thereof.

38. A composition according to claim 37 further comprising at least one sweetener selected from the group consisting of sucrose, fructose, and mixtures thereof.

39. A composition according to claim 38 further comprising a caloric sweetener selected from the group consisting of glucose, dextrose, sucrose, fructose, and mixtures thereof.

40. A composition according to claim 39 comprising less than about 18.5 grams total carbohydrate per every 230 milliliters of the composition.

41. A composition according to claim 40 further comprising one or more beverage components selected from the group consisting of fruit juice, tea, milk solids, and mixtures thereof.

42. A composition according to claim 41 comprising less than about 18 grams total carbohydrate per every 230 milliliters of the composition.

43. A kit comprising:
(a) a composition according to claim 1; and
(b) information that use of the composition is useful for one or more benefits selected from the group consisting of joint health benefits, bone health benefits, anti-inflammation, and utility for diabetic mammals.

44. A kit comprising:
(a) a composition according to claim 13; and
(b) information that use of the composition is useful for one or more benefits selected from the group consisting of joint health benefits, bone health benefits, anti-inflammation, and utility for diabetic mammals.

45. A kit comprising:
(a) a composition according to claim 22; and
(b) information that use of the composition is useful for one or more benefits selected from the group consisting of joint health benefits, bone health benefits, anti-inflammation, and utility for diabetic mammals.

46. A kit comprising:
(a) a composition according to claim 34; and
(b) information that use of the composition is useful for one or more benefits selected from the group consisting of joint health benefits, bone health benefits, anti-inflammation, and utility for diabetic mammals.

47. A method of treating a condition selected from the group consisting of joint dysfunction, bone dysfunction, and inflammation comprising orally administering to a mammal a composition according to claim 1.

48. A method of treating a condition selected from the group consisting of joint dysfunction, bone dysfunction, and inflammation comprising orally administering to a mammal a composition according to claim 13.

49. A method of treating a condition selected from the group consisting of joint dysfunction, bone dysfunction, and inflammation comprising orally administering to a mammal a composition according to claim 22.

50. A method of treating a condition selected from the group consisting of joint dysfunction, bone dysfunction, and inflammation comprising orally administering to a mammal a composition according to claim 34.
